(19) United States
(12) Patent Application Publication
(10) Pub. No.: US 2023/0114161 A1
(43) Pub. Date: Apr. 13, 2023

(54) PERCUTANEOUS RENAL ACCESS SYSTEM

(71) Applicant: JBW7 Innovations, Passaic, NJ (US)

(72) Inventor: Jason B. Wynberg, Passaic, NJ (US)

(73) Assignee: JBW7 Innovations, Passaic, NJ (US)

(21) Appl. No.: 17/860,176

(22) Filed: Jul. 8, 2022

Related U.S. Application Data
(60) Provisional application No. 63/315,578, filed on Mar. 2, 2022.
(60) Provisional application No. 63/254,101, filed on Oct. 9, 2021.
(60) Provisional application No. 63/254,128, filed on Oct. 10, 2021.
(60) Provisional application No. 63/254,503, filed on Oct. 11, 2021.

(51) Int. Cl. A61M 25/06; A61M 25/00

(52) U.S. Cl. CPC A61M 25/0662; A61M 25/0054

(57) ABSTRACT

A method and system for creating a tract in retrograde fashion for nephrostomy tube creation comprising the steps of providing a puncture wire having a tissue penetrating tip shielded in a sheath, inserting the puncture wire and sheath through a channel in an ureteroscope, advancing the puncture wire from the sheath while visualizing under direct vision a position of the puncture wire, advancing the puncture wire through the flank, and inserting a catheter over the puncture wire. In some methods and systems, two catheters are utilized.

This application claims priority to provisional application 63/315,578 filed on Mar. 2, 2022, 63/254,101 filed on Oct. 9, 2021, 63/254,128, filed on Oct. 10, 2021, and 63/254,503, filed on Oct. 11, 2021. The entire contents of each of these applications are incorporated herein by reference.

### BACKGROUND

### Technical Field

This application relates to a percutaneous puncture system and more particularly to a percutaneous puncture system and method for creating a tract for endourologic procedures.

### Background of Related Art

A nephrostomy creation procedure is the creation of a communication between the skin and kidney to provide for endourologic procedure and therapy. The objective in nephrostomy tract creation is to have the wire from outside the flank directed down the ureter to provide therapeutic endourologic management or treatment of a renal system. In the setting of percutaneous endourologic procedures, including but not limited to percutaneous nephrolithotomy, allows for subsequent dilation of the tract, such as by a nephrostomy dilating balloon, between the kidney and the skin over a wire that extends down the ureter. The catheter and tract can also be used to facilitate stenting of a narrowed ureter or removal or treatment of stones obstructing the ureter. Current nephrostomy tract creation is based on x-ray exposure or ultrasound to guide the physician where to locate the nephrostomy puncture wire tract.

Two widely used techniques for nephrostomy tract creation are currently utilized. One technique utilizes an antegrade approach. The antegrade approach holds increased bleeding risk due to the puncture needle puncturing the interlobar arteries as it passes into the collecting system. This antegrade approach is also skill intensive because it requires advancing from the flank to the calyx using two dimensional imaging or targeting aids. In fact, studies have shown that recent urology resident graduates often do not continue to perform the antegrade nephrostomy technique after graduating due to difficulty of this procedure. The fluoroscopy based antegrade puncture procedures require a relatively large amount of radiation exposure.

The other technique commonly utilized is the retrograde puncture technique. This technique is used to create a nephrostomy tract in a retrograde fashion. The original system for this was called the Lawson technique. The Lawson technique is performed under fluoroscopy utilizing a deflecting wire inside a ureteric catheter to select the renal calyx to be entered. That is, fluoroscopy is used to identify the renal calyx for nephrostomy access. The Lawson technique is described for example in Smith's Textbook of Urology, 2007, BC Decker Inc., “Retrograde Access” by Dennis H. Hosking and is commercially available by Cook Urological, Inc. as the “Lawson Retrograde Nephrostomy Wire Puncture Set.”

In the Lawson technique, a stainless steel 145 cm long guidewire (0.038 inches in diameter) having a 3 cm flexible tip is passed retrograde up the ureter into the renal pelvis under fluoroscopy. A 7 Fr catheter is passed over the guidewire into the renal pelvis and the guidewire is removed. A J-tipped wire in certain instances might be used to facilitate passage past an obstruction. Then the surgeon selects the optimal calyx for nephrostomy placement, optimization usually being defined by allowing easiest access to the renal calculi and the shortest tract. Once the calyx is selected, the 0.045 inch diameter deflecting wire guide is inserted through the lumen of the catheter and twist locked to the proximal end of the catheter. Deflection of the wire tip deflects the tip of the catheter, and the catheter and attached wire can be advanced into the selected calyx. However, it is recognized that due to obstructions, e.g., presence of calculi, it may not be possible to advance the catheter into the optimally desired calyx and consequently a less optimal calyx is selected by the surgeon.

After insertion of the catheter into the selected calyx, the deflecting wire guide is removed from the lumen of the catheter, while maintaining the inner-calyx position of the catheter tip. A puncture wire and sheath as a unit are inserted through the catheter lumen, with the puncture wire sharp tip shielded by the sheath. During insertion through the catheter, the wire remains retracted within the sheath, and locked to the sheath by a pin vise lock, so its puncture tip is not exposed. The puncture wire and sheath are connected/locked to the proximal portion of the catheter. The puncture wire is then unlocked from the sheath, by untwisting the cap of the pin vise actuator to loosen the vise pin grip on the puncture wire, and then incrementally advanced from the distal end of the sheath through the flank, fascia and skin. After puncturing the skin, the puncture wire is advanced from below until approximately 15 cm of wire is externally visible.

The pin vise lock securing the puncture wire to the insulating sheath is then re-locked. A fascial incising needle may or may not be passed over the puncture wire at the flank to incise fascia, and is then removed. As the 7 French catheter is advanced through the cystoscope below, the puncture wire is drawn further out of the flank, until the tip of the 7 French catheter is delivered out of the flank. At this time, the 7 French catheter is unlocked from its connection to the puncture wire assembly, and the puncture wire and insulating sheath are removed from below. A 0.038″ guidewire is then passed antegrade through the 7 French catheter from the flank, until it emerges out the lower end of the 7 French catheter at the cystoscope end. With this wire ‘through and through’ the body, the cystoscope and 7 French catheter are removed, leaving the guidewire in place.

The retrograde Lawson approach has several advantages over the antegrade approach including providing the surgeon an anatomic approach to the renal pelvis, increased likelihood of avoiding the interlobar arteries during puncture, and inherently having a wire down the ureter, an important step in securing control over the nephrostomy tract. It is also less skill intensive, due in large part to the fact that it enables travel from the “known kidney” to the “unknown flank/skin,” which better respects the principles of surgery.

However, despite its advantages over the antegrade approach, there are several disadvantages to the Lawson technique. First, it is often difficult to navigate the ureteric catheter beyond large obstructive stones in the renal pelvis. This inability to direct the catheter to the desired site (calyx) often leads the surgeon to access a less optimal calyx. Second, fluoroscopy provides only a two dimensional view of the renal anatomy, thereby limiting the ability to confidently select the calyx for tract dilation. Sometimes, there is even uncertainty as to which calyx is actually chosen due to the limited visibility provided by fluoroscopy.

Consequently, it would be advantageous to provide a system and method that enables more precise calyx location, improves access to the calyx of choice, improves visualization, permits “fluoroscopy-free” calyx selection, and allows for preliminary laser lithotripsy of a portion of a stone that may block access to calyx of choice for nephrostomy creation. Also of significance is that nephrostomy tube creation procedures are usually performed by interventional radiologists, which can further compound the risks and problems since urologists usually have better success rates for selecting the calyx for such procedures. Thus, it would be advantageous if such improved system and method could be more commonly performed by urologists.

In an attempt to address some of the disadvantages of the Lawson technique, Dr. Larry C. Munch in an article entitled “Direct-Vision Modified Lawson Retrograde Nephrostomy Technique Using Flexible Ureteroscope” and published in the Journal of EndoUrology, Volume 3, Number 4, 1989, described a technique utilizing a flexible ureteroscope.

In this “Munch technique,” a flexible steerable ureteroscope was utilized to inspect the renal pelvis and calices. As described, a flexible cystoscopy is performed and a 0.035 inch, 145 cm guidewire is passed into the ureteral orifice. Position within the ureter is assessed with fluoroscopy. The cystoscope is removed and a ureteral access sheath with its obturator is advanced over the guidewire, and the obturator is then removed and the ureteroscope is passed through the sheath into the renal pelvis. An appropriate calyx is chosen visually, and then the 0.0017 inch Lawson puncture wire and protective 3Fr radiopaque Teflon sheath is passed through the working channel of the ureteroscope. The calyx is entered and the sheath embedded in the wall of the calyx, and then the pin-vise lock which locks the puncture wire and sheath together is opened and the puncture wire is advanced through the skin under visual and fluoroscopic control. The puncture wire protective sheath and ureteroscope are then withdrawn, leaving the puncture wire and ureteral access sheath in place. At the skin, an 18 gauge needle is passed over the puncture wire into the kidney and then removed. A 9 French fascial dilator is then passed over the 0.017 inch puncture wire into the kidney, whereafter the puncture wire is removed and a 0.038 inch guidewire is passed through the 9 French dilator until it passes down the ureter through the access sheath, and exits through the urethra. Thus, Munch was the first to describe retrograde nephrosotomy via retrograde puncture from “inside to outside” though a flexible ureteroscope pre-positioned in a renal calyx.

Although the Munch technique solves some of the problems associated with the Lawson technique, it still has several deficiencies. Munch's technique of antegrade wire exchange is ineffectual and risks cutting the puncture wire with passage of 18 gage hollow bore needle over the wire. After passage of this needle, a 9 French fascial dilator is passed over the 0.017″ puncture wire, representing a wire-catheter mismatch which can result in tearing of internal tissues. This large jump from an 18 gauge needle to a 9 French fascial dilator is also cumbersome and has a high chance of failing to grant access to the kidney. In addition to employing a wire that could both kink during regular use and “shear” through the kidney and ureteropelvic junction if exposed due to wire thickness, Munch's wire exchange system has been ineffectual, with a reported use of a rigid needle cannula which could not direct the wire into the ureter.

Consequently, it would be advantageous to provide a system and method that would enable urologists to more economically and efficiently perform the nephrostomy procedure to obtain access for nephrostomy tube creation. Such procedure would have the above-noted advantages over the Lawson technique, e.g., improving calyx access, visualization etc., while also providing the advantages of reducing the number of surgical steps and reducing the possibility of tearing tissue, thereby providing advantages over the Munch technique.

In U.S. Pat. Nos. 8,771,287 and 8,888,787 (common inventor and owner as the present application), systems and methods are disclosed which provide improvements to the foregoing deficiencies and overcome the disadvantages of Lawson, Munch and other prior systems. The systems of these patents use a coaxial catheter to effect wire exchange as well as a dual diameter puncture wire to provide a thicker portion of wire over the ureteropelvic junction if exposed to this anatomic region. These features are incorporated into the commercialized RetroPerc* Flexible Ureteroscopy Guided Retrograde Nephrostomy Wire Puncture Set. In this setting, the puncture wire performs both a) renal puncture and b) serves as a bridge over which the wire exchange catheters are advanced at the flank, where the tip protector sheath over the puncture wire does not emerge from the flank. The RetroPerc* system employs a coaxial microintroducer advanced over the puncture wire at the flank to permit placement of a larger diameter second nephrostomy wire. These systems have several advantages which are described in these patents.

The inventor of these systems recognized that alternative systems could be utilized to achieve the same and in fact additional advantages, and conceived and designed unique variations which enable endourologic procedures.

### SUMMARY

The present invention provides methods of wire exchange at the flank, over an emergent puncture wire for nephrostomy tube creation. Each of the two systems/methods of the present invention employ single lumen exchange catheters and are described in detail herein. The catheters are configured and dimensioned to facilitate wire exchange and ureter access. One system utilizes a single catheter; another system utilizes two catheters. The catheters are sized to accommodate standard sized puncture wires and endourology working wires, and the methods utilize such sizing to obtain desired access.

The present invention also provides in some embodiments a puncture wire construction which advantageously reduces kinking of the wire. This is discussed in detail below.

In accordance with one aspect of the present invention, a method for creating a tract in retrograde fashion for nephrostomy tube creation is provided comprising the steps of:
a) inserting a puncture wire and sheath in a first direction through a working channel in an ureteroscope to exit the working channel, the puncture wire having a tissue penetrating tip shielded in the sheath;
b) advancing the puncture wire a first distance from the sheath into a selected papilla and through a flank of a patient, the puncture wire having an emergent segment extending beyond the flank of the patient;
c) advancing a catheter over the emergent segment of the puncture wire outside the flank in a second direction opposite a direction of renal and flank puncture;
d) advancing the catheter into the flank in the second direction, the catheter having a lumen having a first section and a second section at a distal tip of the catheter, the first section having a first luminal diameter and the second section having a second lumenal diameter smaller than the first diameter;
e) removing the puncture wire from the catheter; and
f) advancing a second wire in the second direction through the catheter into a ureter of the patient, the second wire having an outer diameter greater than an outer diameter of the puncture wire and greater than the second luminal diameter of the second section of the lumen.

In some embodiments, the puncture wire is slidable within the sheath and releasingly lockingly engageable therein, and the puncture wire is released from the sheath prior to the step of advancing the puncture wire.

In some embodiments, the second wire expands the distal tip of catheter when passed through the second section of the lumen.

In some embodiments, the puncture wire has a proximal segment with an outer diameter larger than a distal segment of the puncture wire to add stability to manual advancement of the catheter and prevent kinking of the puncture wire during advancement of the catheter. In some embodiments, the distal segment has a larger diameter than a distalmost puncture point of the penetrating tip. In some embodiments, the proximal segment is positioned outside an entry point to the ureteroscope when the puncture wire is inserted into the ureteroscope.

In some embodiments, the puncture wire has a distalmost puncture point, a first segment adjacent and proximal of the distalmost puncture point and a second segment proximal of the first segment, wherein the first segment has an outer diameter larger than an outer diameter of the puncture point and smaller than an outer diameter of the second segment to allow flexion of the first segment, and the second segment serves as a bridge over which the catheter is passed and limits kinking of the puncture wire during advancement of the catheter thereover.

In some embodiments, the puncture wire has a proximal segment with an outer diameter larger than a distal segment to prevent kinking during both advancement of the puncture wire through the ureteroscope and advancement of the catheter over the puncture wire in the second direction opposite the direction of advancement through the ureteroscope.

In some embodiments, the second section of the lumen of the catheter has little or zero clearance over the puncture wire.

In accordance with another aspect of the present invention, a method for creating a tract in retrograde fashion for nephrostomy tube creation is provided comprising the steps of:
a) inserting a puncture wire having a tissue penetrating tip shielded in a sheath in a first direction through a working channel in an ureteroscope to exit the working channel;
b) advancing the puncture wire a first distance from the sheath through a flank of a patient so the puncture wire has an emergent segment extending beyond the flank of the patient;
c) advancing a first single lumen catheter over the emergent segment of the puncture wire outside the flank in a second direction opposite a direction of renal and flank puncture and advancing the first catheter into the flank to dilate tissue around the puncture wire, the first catheter having a distal tip having an inner diameter being a zero or close to zero fit over the puncture wire to ease passage into flank tissues;
d) removing the first catheter;
e) advancing a second single lumen catheter over the emergent section of the puncture wire outside the flank and advancing the second catheter into the flank and ureter in the second direction opposite the direction of renal and flank puncture;
f) removing the puncture wire leaving the second catheter in position in the ureter and outside the flank; and
g) advancing a second wire through the second catheter, the second wire having an outer diameter larger than an outer diameter of the puncture wire, wherein the second catheter has an inner diameter larger than the inner diameter of the first catheter.

In some embodiments, the second wire has an outer diameter larger than the inner diameter of the first catheter.

In some embodiments, the second catheter has an inner diameter substantially equal to the outer diameter of the second wire.

In some embodiments, the puncture wire is slidable within the sheath and releasingly lockingly engageable therein and the puncture wire is released from the sheath prior to the step of advancing the puncture wire.

In accordance with another aspect of the present invention, a system for creating a tract for nephrostomy tube creation is provided comprising:
a) an exchange catheter having a tapered end forming a reduced diameter tip region, the tip region expandable by passage of a wire having a larger outer diameter than the reduced diameter tip region;
b) a protective sheath; and
c) a puncture wire slidable within the sheath and releasably lockingly engageable with the sheath, the puncture wire having a puncture tip and a thicker proximal segment at a point of advancement by a clinician above and outside an entry to a working channel of a ureteroscope, the proximal segment thicker than a distal segment adjacent the puncture tip, the thicker segment adding stability to advancement of an exchange catheter thereover and preventing kinking during advancement of the exchange catheter thereover.

In some embodiments, the system further comprises a second wire having an outer diameter larger than an outer diameter of the proximal segment of the puncture wire and larger than an inner lumen of the reduced diameter tip region to expand the reduced diameter tip region when received therethrough.

In some embodiments, the second catheter has an inner diameter equal to or larger than an outer diameter of the second wire.

### BRIEF DESCRIPTION OF THE DRAWINGS

So that those having ordinary skill in the art to which the subject invention appertains will more readily understand how to make and use the surgical systems disclosed herein, preferred embodiments thereof will be described in detail hereinbelow with reference to the drawings, wherein:

FIG. 1 is a perspective view of a first embodiment of the puncture wire, pin vise lock and sheath of the present invention;

FIG. 1A is a perspective view of an alternate embodiment of the puncture wire, pin vise lock and sheath of the present invention;

FIG. 1B is a perspective view of an alternate embodiment of the puncture wire of the present invention having a narrower distalmost segment and a beveled tip;

FIG. 1C is a perspective view of an alternate embodiment of the puncture wire of the present invention having a narrower distalmost segment and a pencil-point tip;

FIG. 1D is a perspective view an alternate embodiment of the puncture wire of the present invention having a pencil point tip;

FIG. 1E is side view of an endourology wire having a larger diameter than the puncture wire of FIG. 1D, the two wires shown side by side;

FIG. 1F is a side view of an embodiment of the single lumen dilator/catheter of the present invention having a tapered tip forming a reduced diameter tip region;

FIG. 1G is a side view of two catheters of different diameters for use in the two-catheter method of the present invention;

FIG. 2 is a side view illustrating the pin vise lock of FIG. 1 for locking the sheath and puncture wire together;

FIG. 3 illustrates initial insertion of a puncture wire and sheath of one embodiment through a working channel of a flexible ureteroscope positioned through the ureter and extending into the kidney, for clarity the ureteral access sheath is not shown;

FIG. 4 illustrates the ureteroscope inserted through a selected calyx and the sheath and puncture wire being advanced in a first direction through the ureteroscope;

FIG. 5 is a view similar to FIG. 4 showing a portion of the ureteroscope of FIG. 1 and showing further insertion of the puncture wire and sheath in a first direction through the ureteroscope with the sheath and puncture wire extending distal of the ureteroscope;

FIG. 5A is a view similar to FIG. 5 illustrating the puncture wire advanced in a first direction through the flank and skin;

FIG. 6 is a view similar to FIG. 3 corresponding to the position of the puncture wire and sheath of FIG. 5A;

FIG. 7 is a close-up view of one embodiment of a locking mechanism for the puncture wire and sheath;

FIG. 8 is a view similar to FIG. 7 showing rotation of the knob to lock the puncture wire and sheath;

FIGS. 9-14 illustrate one embodiment of the method of the present invention using one single lumen catheter wherein:

FIG. 9 is a view similar to FIG. 5A illustrating the puncture wire further extended through the skin;

FIG. 10 is a view similar to FIG. 9 showing initial insertion of the single lumen tapered catheter of FIG. 1F inserted in a second direction (antegrade) over the exposed (emergent) segment of the puncture wire of FIG. 1D;

FIG. 11 is a view similar to FIG. 10 showing insertion of the catheter of FIG. 1F over the puncture wire into the ureter and further showing the puncture wire held by a clamp;

FIG. 11A is a close-up view showing the catheter closely fitting over the puncture wire;

FIG. 12 is a view similar to FIG. 11 showing the puncture wire removed leaving the catheter in place;

FIG. 13 is a view similar to FIG. 12 showing advancement of the larger diameter second (working) wire of FIG. 1E in the second direction (antegrade) through the open end of the catheter exposed at the flank;

FIG. 13A is a close-up view showing the second wire being inserted through the catheter of FIG. 13;

FIG. 13B is a close-up view similar to FIG. 13A showing the catheter tip expanded by passage of the second wire;

FIG. 14 is a view similar to FIG. 13 showing removal of the catheter in a direction opposite the direction of its initial insertion;

FIGS. 15-21 illustrate another embodiment of the method of the present invention utilizing two single lumen catheters wherein:

FIG. 15 is a view similar to FIG. 10 showing initial insertion of the smaller single lumen tapered catheter of FIG. 1G inserted over the exposed (emergent) segment of the puncture wire of FIG. 1D;

FIG. 16 is a view similar to FIG. 15 showing insertion of the catheter in a second direction (antegrade) over the puncture wire into the ureter to dilate the tissue around the puncture wire (the puncture wire held by a clamp);

FIG. 17 is a view similar to FIG. 16 showing removal of the catheter in a direction opposite its direction of insertion;

FIG. 18 is a view similar to FIG. 17 showing advancement of a second larger diameter catheter of FIG. 1G in the second direction over the puncture wire into the ureter;

FIG. 19 is a view similar to FIG. 18 showing removal of the puncture wire removed leaving the second catheter in place;

FIG. 20 is a view similar to FIG. 19 showing insertion of the second larger diameter (working) wire of FIG. 1E through the second catheter; and

FIG. 21 is a view similar to FIG. 20 showing removal of the second catheter in a direction opposite the direction of its initial insertion to leave the second working/endourology wire in place;

FIG. 22 is a perspective view of one embodiment of a kit of the present invention having a puncture wire within a protective sheath along with the single lumen catheter of FIG. 1F; and

FIG. 23 is a perspective view of another embodiment of a kit of the present invention having a puncture wire within a protective sheath along with two of the single lumen catheters of FIG. 1G.

### DETAILED DESCRIPTION OF PREFERRED EMBODIMENTS

The present invention provides methods of wire exchange at the flank, over an emergent section of the puncture wire section of the puncture wire extending from the flank and skin. In general, two systems/methods are disclosed, both utilizing a puncture wire and a protective sheath. Each of the two systems employ single lumen exchange catheters and are described in detail below. It should be appreciated that multiple lumen catheters could also be utilized provided they meet the dimensional aspects advantages of the single lumen catheters as used in the methods disclosed herein.

In the first system described herein, a single lumen catheter/dilator is used for insertion of an endourology working wire. This simplifies the components and procedural steps of the surgery. In the second system described herein, two single lumen catheters are utilized: a first catheter dilates the tissue around the puncture wire to ease passage of the second catheter over the puncture wire, the second catheter then provides for passage of the larger endourology working wire. More specifically, in the first system, a single lumen catheter is passed over the emergent puncture wire, followed by removal of the puncture wire and insertion of a larger wire, followed by removal of the single lumen catheter leaving the larger wire in place. In the second system, two single lumen catheters are utilized wherein a first catheter is passed over the puncture wire, followed by removal of the first catheter, then insertion of a second larger catheter over the puncture wire, and then removal of the puncture wire and advancement of a second wire into the second catheter, followed by removal of the second larger catheter, leaving the second wire in place.

The present invention also provides in some embodiments a puncture wire construction which advantageously adds stability to advancement of an exchange catheter thereover and/or reduces kinking of the wire. This is also discussed in detail below.

The present invention provides accessing as well as selecting a calyx under direct visualization utilizing an ureteroscope in order to create a nephrostomy tract.

In the present invention, a puncture wire is advanced through a working channel of an ureteroscope which has been passed into the kidney in retrograde fashion. The puncture wire is then deployed from the ureteroscope working channel through a surgeon selected calyx and through the kidney and out the flank and skin in a retrograde fashion. This technique obviates the need for antegrade access to the calyx as antegrade access disadvantageously requires significant technical skill due to limited targeting systems which typically function in two dimensions and creates potential risks for the patient including relatively high radiation exposure. This retrograde approach provides improved three-dimensional endoscopic targeting and protects the renal anatomy to prevent tissue damage during the procedure.

Referring now in detail to the drawings wherein like reference numerals identify similar or like components throughout the several views, various embodiments of the present invention are disclosed.

As used herein the term “proximal” denotes the region closer to the user and the term “distal” denotes the region further from the user.

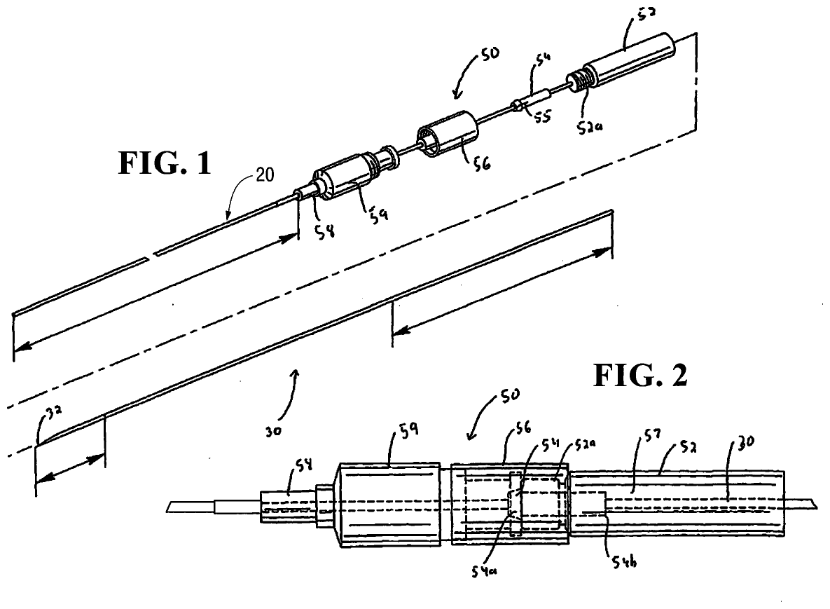

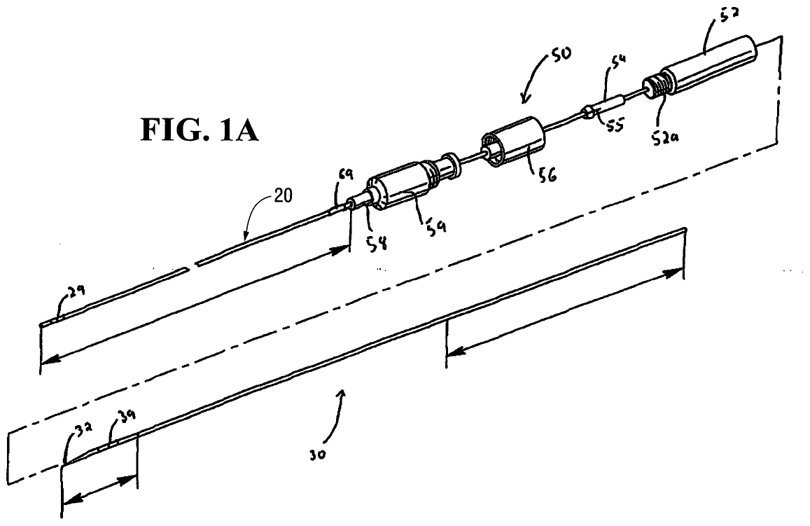

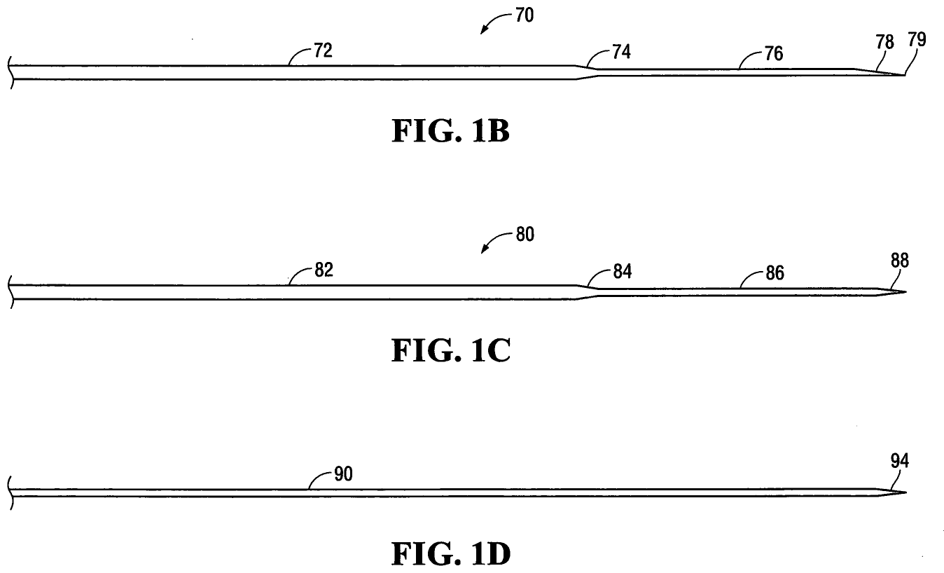

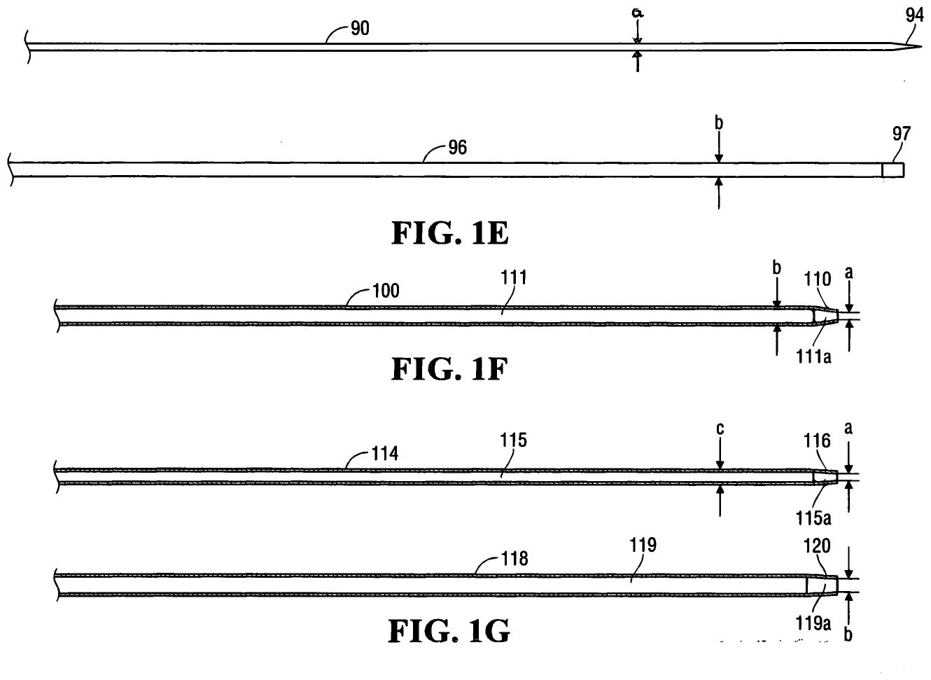

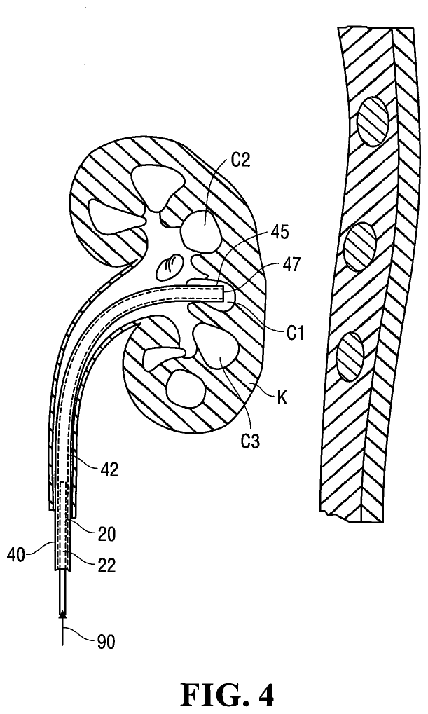

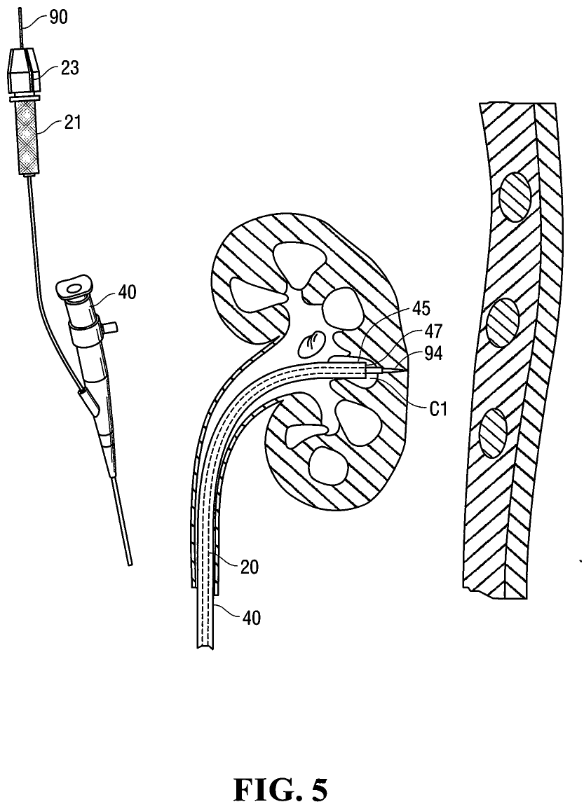

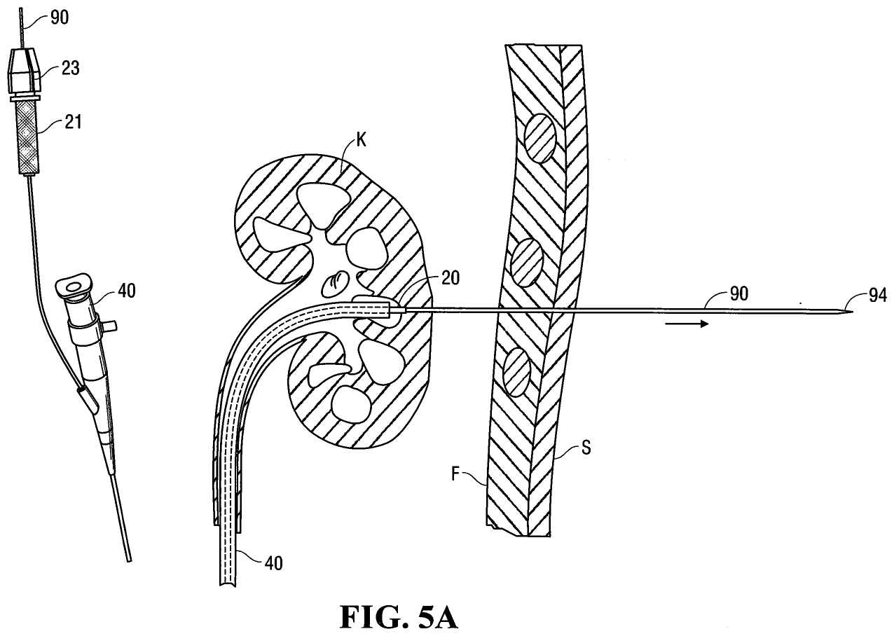

With reference to FIGS. 1, 1A and 2, like the system of U.S. Pat. No. 8,888,787, the system of the present invention includes a protective sheath 20 and a puncture wire 30. Sheath 20 has a lumen 22 extending therethrough dimensioned to slidingly receive the puncture wire 30 therein. That is, puncture wire 30 is received within the sheath lumen 22 for sliding movement from a retracted position wherein the puncture (penetrating) tip 32 of wire 30 is protected (shielded) by the sheath 20 (like the puncture wire of FIG. 4) and an extended position where the puncture tip 32 is exposed from the sheath 20 to penetrate tissue (like the puncture wire of FIG. 5) as the puncture tip 32 extends beyond the distal opening of the sheath 20. Exposure of the puncture wire tip 32 enables advancement of the wire 30 through the flank and skin as described below.

The sheath 20 preferably has a length of between about 70 cm to about 170 cm, and more preferably between about 85 cm to about 115 cm. With this length, the sheath 20 has sufficient length for insertion through the entire working channel 42 of the ureteroscope 40, which typically has a length of approximately 85-95 cm including the portion of channel within the ureteroscope handle. The sheath is preferably a 2.5-2.7 French sheath, having an internal diameter that is sufficient to receive the puncture wire. Other dimensions are also contemplated such as sheath diameters of between about 0.038 inches and about 0.07 inches. The sheath is preferably composed of PTFE (e.g., polyimide or similar), although other materials are also contemplated. The sheath can be constructed with braiding or high durometer materials.

The puncture wire 30 and sheath 20 are releasably locked together by a conventional vise lock 50. FIGS. 1 and 2 show one example of a sheath locking mechanism. Vise lock 50 has a rotatable actuator 52 and a metal locking tube 54 with a longitudinally extending elongated slot. A first (distal) portion 54a of locking tube 54 is seated within tube 56; a second opposite proximal portion 54b is seated within the actuator. Actuator 52 has a reduced diameter portion 52a threadingly received in tube 56 and a lumen 57 through which the puncture wire 30 extends. When actuator 52 is rotated within tube 56, it clamps down on the metal locking tube 54 reducing its diameter due to the slot, to thereby clamp down on the puncture wire 30 to lock it from sliding movement with respect to the sheath 20. A reinforcement tube 58 extends distally from distal tube 59 which can connect via screw threads (or by other methods).

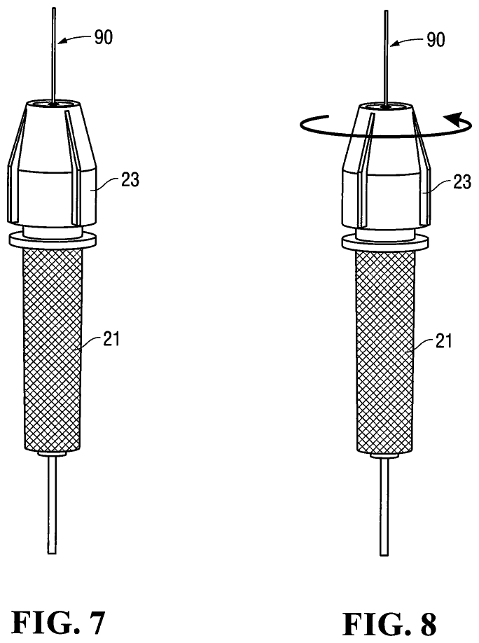

FIGS. 7 and 8 illustrate an alternate embodiment of a pin vise lock for locking the puncture wire and sheath together. Instead of the three piece system of FIG. 1, a two piece system is provided wherein the clamping feature is within tubular portion 21 and rotation of actuator (knob 23), effects clamping down on the puncture wire via clamping down on an internal locking tube similar to tube 54 of FIG. 2. As described below with respect to the method of use, the puncture wire and sheath 20 can be locked together so they can be advanced as a unit through the ureteroscope. When it is desired to move the puncture wire relative to the sheath 20, the actuator 52 or 23 is unscrewed or rotated in a reverse direction, thereby releasing the clamping force on the internal metal tube so the puncture wire can slide relative to the sheath 20.

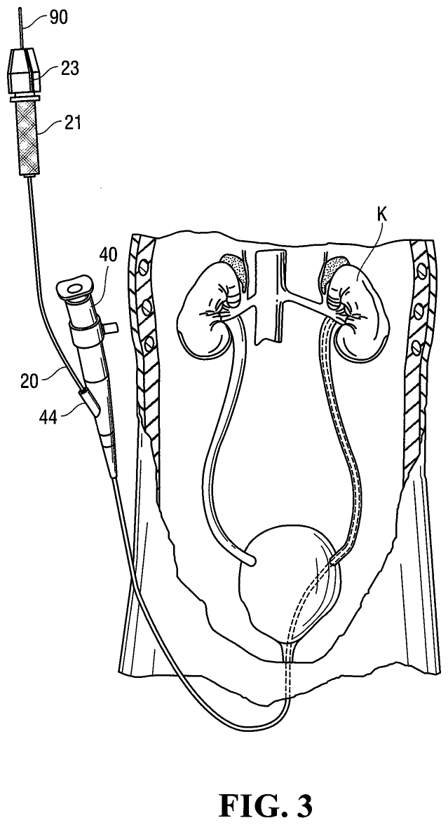

Note the locking mechanism depicted in FIGS. 1 and 2 illustrates puncture wire 30 and the locking mechanism depicted in FIGS. 3, 7 and 8 illustrates puncture wire 90. It should be appreciated that the locking mechanisms disclosed herein, as well as alternative locking mechanisms, can be utilized with any of the puncture wires disclosed herein to lock together the sheath and puncture wire.

The system also can also in some embodiments include a sheath locking mechanism, such as a circumferentially tightening O-ring mechanism for locking the sheath 20 to a working channel of the ureteroscope 40 as described in U.S. Pat. Nos. 8,888,787, and 8,771,287, the entire contents of each of these patents incorporated herein by reference.

The puncture wire 30 of FIG. 1A has a beveled tip. Various alternate puncture wire configurations are disclosed in FIGS. 1B-1D. In FIG. 1D, the puncture wire 90 is similar to puncture wire 30 except it has a pencil point tip 94 rather than a beveled tip. Puncture wire 90 in this embodiment has a uniform diameter along its length, from a proximal end to a distal end except for the distal tip which narrows to form a sharp pencil point tip 94. An increased diameter proximal portion/segment can be provided to reduce or prevent kinking of the portion/segment of the wire outside the ureteroscope during advancement of the wire and at the location of advancement of the exchange catheter.

The puncture wire 80 of the alternative embodiment of FIG. 1C has a pencil-point tip 88 as in wire 90, except it has changing diameters along its length. As shown, it has two diameters, although additional diameters are also contemplated. First, a narrower distalmost segment (portion or region) of the puncture wire 80 has a distalmost puncture point 88. The segment (portion or region) 86 just proximal to the sharp point 88 has a narrower diameter to allow flexion of the distal segment of the flexible ureteroscope while the puncture wire and sheath are inside the working channel of the flexible ureteroscope. The segment (portion or region) 82 of the wire 80 which is proximal to the distal segment 86 has a larger diameter than the distal segment 86. (Reference numeral 84 designates the diameter transition region).

The segment 82 of wire 80 can have a uniform diameter or have an increased diameter at a more proximal region. The segment 82 serves as a bridge over which an exchange catheter is passed. This segment 82 being thicker prevents kinking of the wire 80 during advancement of the exchange catheter from “outside to inside” over the wire. This segment 82 is also thicker than the distal segment 86 of the puncture wire 80 and prevents kinking of the exposed segment above and outside the ureteroscope during advancement. The proximalmost segment of wire 80 can be of the same diameter as the remaining segment 82 or can be of a larger diameter to provide an even thicker kink preventive segment. The proximalmost segment could also in alternative embodiments be a little smaller in diameter that the segment 82 but larger than the diameter of segment 86. The proximalmost segment of the puncture wire 80 is exposed outside the flexible ureteroscope as shown for example in FIG. 3.

FIG. 1B illustrates another alternate embodiment of the puncture wire of the present invention. Wire 70 is similar to wire 80 of FIG. 1C as it has a larger diameter region 72 transitioning at transition region 74 to a smaller diameter region 76. Wire 70 differs from wire 80 of FIG. 1C in that it has a beveled distal end 78 with a sharp point 79 rather than a pencil point. Wire 70, like the other wires disclosed, herein can have proximal segments of increased diameter to reduce kinking.

The puncture wire 90 (as well as the other puncture wires disclosed herein) has a diameter less than a diameter of the endourology working wire, also referred to herein as the replacement wire. This can be appreciated by reference to FIG. 1E showing the puncture wire 90 by way of example, with an outer diameter “a”, next to a working wire 96 with an outer diameter “b” larger than outer diameter “a”. The tip 97 of wire 96 can also have a diameter equal to or larger than diameter “a.” By way of example, diameter “a” could be in the range of about 0.013 inches to about 0.027 inches, and preferably about 0.019 inches, and diameter “b” could be in the range of about 0.025 to about 0.042 inches and preferably about 0.035 inches or 0.038 inches. Other diameters are also contemplated. The diameters of wire 90 are smaller than an internal diameter of the protective sheath 20 to allow sliding movement within the sheath.

It should be appreciated that puncture wire 90 is shown side by side with replacement wire 96 (also referred to herein as the endourology wire, working wire or the second wire) by way of example. The other puncture wires 30, 70 and 80 would also preferably have an outer diameter in the ranges set forth above (diameter “a”) and function in the same manner as puncture wire 90. Also, as should be appreciated, in the methods described below, puncture wire 90 is described and shown by way of example, it being understood that puncture wire 30, 70 and 80 can also be used in the same manner as puncture wire 90.

A conventional ureteroscope 40 is shown in FIG. 3 and includes a working (operating) channel opening communicating with an internal channel (lumen) 42 (see e.g., FIG. 4) forming a working lumen for insertion of the puncture wire and sheath. The ureteroscope 40 is preferably a steerable scope so it can be articulated through the urinary system to gain access to the desired calyx. The working channel 42 is accessible through an opening in a port or hub, e.g., through a side arm, which communicates with the ureteroscope channel 42 extending longitudinally within the length of the ureteroscope 40. The ureteroscope 40 provides both illumination and visualization of the surgical site as well as illumination and visualization of the puncture wire and sheath 20 as they are advanced from the distal opening 47 of the ureteroscope 40, thus providing visualization of the system components as well as the patient's body. The ureteroscope 40 typically has a working channel length of about 55 cm to about 75 cm plus a portion of working channel length within the handle of ureteroscope of about 10 cm to about 25 cm (total working channel length about 75 cm to about 100 cm), a total outer diameter at the tip typically of about 5 French to about 8.1 French, with a working channel diameter of about 3 French to about 4.5 French. The working channel 42 is also dimensioned to receive a laser fiber for reducing blocking stones as described below. The ureteroscope is preferably inserted through a ureteral access sheath (not shown).

The puncture wire 90 preferably has a length of between about 130 cm to about 185 cm, and more preferably a length of about 160 cm. With this length, the puncture wire 90 has sufficient length for insertion through the entire working channel 42 of the ureteroscope 40 as well as sufficient length to exit therefrom and extend through the flank and skin. The puncture wire can be composed of stainless steel, although other materials are also contemplated. Note that other wire lengths and diameters are also contemplated.

The puncture wires of the FIGS. 1A-1D can in some embodiments have one or more markings on their outer surface to indicate to the surgeon its position with respect to the sheath 20, skin, and/or ureteroscope 40. The markings can be placed on a region of the puncture wire extending outside the body or alternatively or additionally on a region extending within the body to be imaged by the ureteroscope 40. Likewise, the sheath 20 can have one or more markings on a region outside the body, and/or inside the body where the marking(s) can be visualized by the ureteroscope 40. FIG. 1A illustrates markings 39 on a distal portion of puncture wire 30 and markings 29 on a distal portion of sheath 20 by way of example.

As noted above, there are two different systems/methods of the present invention. One system (referred to herein as the “first” system solely for convenience) includes a single lumen dilator catheter 100 with a lumen 111 as shown in FIG. 1F. At the distal tip 110 of the catheter 100 is a reduced diameter flexible region which has lumen 111a which is smaller than lumen 111. That is, the distal end of lumen 111 tapers in a distal direction to lumen 111a having a smaller diameter. The OD of the distal tip 110 also tapers to a reduced diameter. Stated another way, lumen 111 has a first section and a second more distal section at the distal tip having a smaller diameter than the first section. The first and second sections can each be of uniform diameter or of changing diameters provided the lumen at the distal tip has the smaller diameter than more proximal regions. In preferred embodiments, the inner diameter of catheter 100 is large enough to accommodate a second replacement/endourology/working wire such as wire 96 of FIG. 1E. The outer diameter of the distal tip 110 of the catheter 100 can in some embodiments be smaller than the outer diameter of the second wire 96 to ease passage into the flank tissues over the narrower (smaller diameter) puncture wire.

In the illustrated embodiment, the catheter 100 has an inner diameter “a” at the distal tip which is the same (or substantially the same) as the outer diameter “a” of the puncture wire 90. Catheter 100 has an inner diameter “b” which is greater than the outer diameter “a” of the puncture wire and equal to (or slightly larger than) the outer diameter “b” of working wire 96 as shown in FIG. 1E. In this manner, puncture wire 90 does not stretch the distal tip 110 while working wire 96 does stretch tip 110 due to its smaller inner diameter. Thus, the lumen of the catheter 100 accepts the larger working wire 96 as well as the puncture wire 90, and the distal tip 110 of catheter 100 is tapered to a diameter of the smaller diameter puncture wire such that the puncture wire does not expand/stretch the distal tip 110. Note the puncture wire would fit loosely within the lumen 111 of the catheter 100 proximal to lumen 111a.

In the first system, the single lumen dilator/catheter in preferred embodiments is about 12 cm to about 50 cm in length, and preferably about 20 to about 40 cm. This catheter preferably has an inner diameter of at least 0.025″ and less than 0.055″. It could also have an inner diameter between about 0.035″ and about 0.045″ to accommodate the larger diameter second endourology working wire after removal of the narrower puncture wire from within.

Note FIG. 1F shows a single catheter used in one method of insertion utilizing one catheter. FIG. 1G illustrates two catheters for use in an alternative system/method. This two-catheter system is referred to herein as the “second” system for convenience. The two catheters 114, 118 are of different lumenal and tip diameters. Catheter 114 has a lumen 115 tapering at distal tip 116 to a smaller lumen 115a. Thus, the lumen 115 has a first section and a second more distal section 115a at the distal tip having a smaller diameter than the first section. The internal diameter “a” at tip 116 (lumen 115a) has a diameter matching or substantially the same as the outer diameter “a” of wire 90. Catheter 114 has an internal diameter “c” which is less than the outer diameter “b” of the working wire 96 but greater than outer diameter “a” of the puncture wire 90 (and for puncture wire 70, greater than the outer diameter proximal to segment 76, and for puncture wire 80, greater than the outer diameter proximal to segment 86). Thus, catheter 114 has a snug fit over wire 90 (or wire 30, and for puncture wire 70, a snug fit proximal to segment 76, and for puncture wire 80, a snug fit proximal to segment 86). In other words, the catheter 114 has a lumen large enough to pass over the puncture wire 90 but less than the outer diameter of the working wire 96 so it cannot receive the working wire 96.

The second catheter of the second system, designated by reference numeral 118, has a larger inner diameter and larger outer diameter than catheter 114. Catheter 118 has a lumen 119 at distal tip 120 tapering distally to a reduced diameter lumen 119a at the second section of the lumen at the distal tip. The inner diameter at tip 120, i.e., the diameter of lumen 119a, at the distalmost entry into the catheter 118 has a diameter “b” which matches or substantially matches the diameter “b” of the working wire 96 so the second catheter 118 is large enough to pass over the larger second wire 96.

Thus, in this two catheter embodiment, while the inner diameter of the first catheter 114 is smaller than the outer diameter of the larger wire 96, the outer diameter of the first catheter 114 is larger than the outer diameter of the second wire 96. This means that the catheter 114 passes snugly over the thin first puncture wire 90 and its outer diameter will stretch fascia larger than the second wire 96 so that when the second catheter 118 is loaded over the puncture wire 90, the clearance/space between the puncture wire outer diameter and the tip inner diameter won't prevent advancement as the tissues will have been stretched already.

Note the size/diameter relationship is discussed above with respect to puncture wire 90, but is also applicable to the other puncture wires disclosed herein.

Turning now to the method of use, and with reference to FIGS. 3 and 4, the ureteroscope 40 is inserted through the ureter U and extends up to the kidney K. This is applicable to both the “first” and “second” methods. The ureteroscope 40 is manipulated under vision so its distal end 45 extends into the calyx of choice, e.g., calyx C1 (FIG. 4). Note the ureteroscope 40 can be articulated into the calyx of choice. Note the methods described below utilizes puncture wire 90; however, it should be appreciated that other puncture wires, e.g., puncture wire 30, 70 or 80 can also be used in the same manner as puncture wire 90 and thus the discussion below of the methods of use are fully applicable to use with puncture wires other than the illustrated puncture wire 90.

If during insertion of the ureteroscope 40 a stone is encountered under visualization that is blocking the path to the desired calyx C, e.g., calyx C1, C2, C3 etc., a laser fiber (not shown) can be inserted through the working channel 42 of the already positioned ureteroscope 40 to perform laser lithotripsy to reduce the size of the stone to allow access by the ureteroscope 40 to the desired calyx. The laser fiber can then be removed from the working channel 42.

After placement of the ureteroscope 40 at the desired location, e.g., into calyx C1 of FIG. 4, the puncture wire 30 and sheath 20, locked together by tightening of the pin vise lock mechanism 23 via rotation of knob 21 as described above, are inserted through the working channel 42 of ureteroscope 40. This initial insertion is illustrated in FIGS. 3 and 4. At this point, the puncture wire tip 94 of puncture wire 90 is retracted and thereby shielded within the protective sheath 20.

The puncture wire 90 and sheath 20 are then advanced in the first direction through the working channel exiting just distal of the tip 45 of the ureteroscope 40 (beyond distal opening 47 as shown in FIG. 5), and viewed to make sure they are in the desired anatomical position.

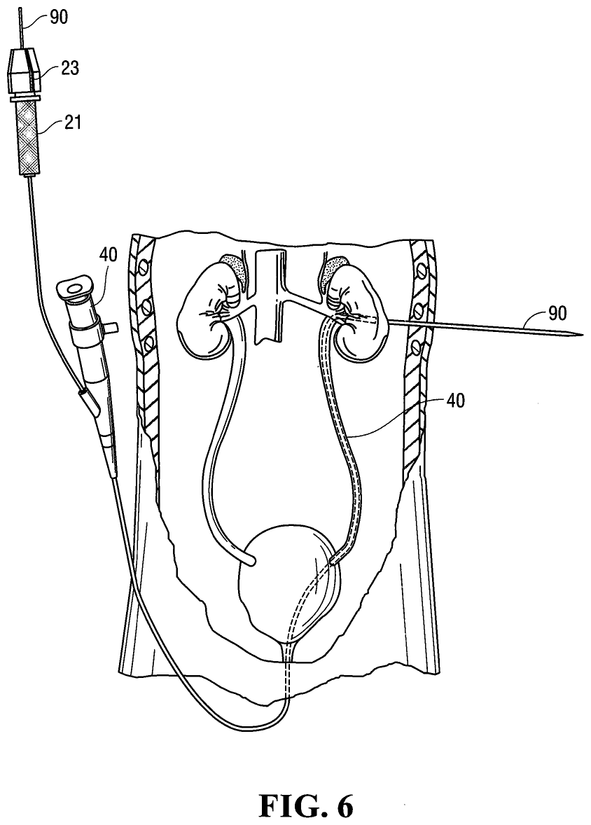

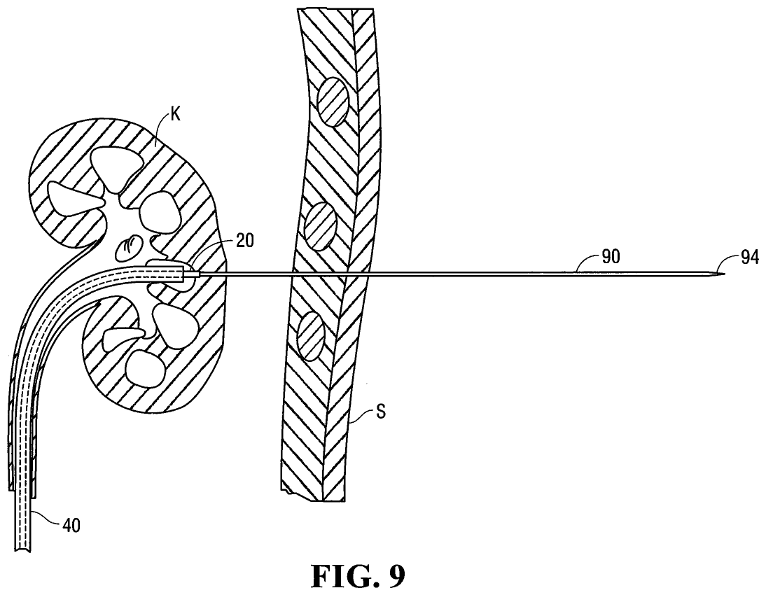

To next advance the puncture wire 90 further through the scope 40 and sheath 20, actuator 23 of pin vise lock 21 is rotated as described above, thereby releasing the locking engagement of the puncture wire 90 and sheath 20. This enables advancement of the puncture wire 90 (see arrow in FIG. 5A) through the kidney K, selected papilla, flank F and skin S as shown in FIGS. 5A and 6. The puncture wire 90 is advanced further in the first direction (retrograde) to a position shown in FIG. 9, with the sheath 20 remaining in the position of FIGS. 5A and 6 and an “emergent” section of the puncture wire exposed. Note that the puncture wire 90 is protected along its length by the sheath 20 as well as by the ureteroscope 40 during insertion, prior to exposure from the sheath in FIG. 5A.

It should be appreciated that alternatively the sheath 20 and puncture wire 90 can be locked together by the pin vise locking mechanism 50, with the puncture tip 94 slightly protruding from the sheath 20, and advanced together through the flank tissues rather than only the puncture wire 30 being advanced through the flank tissues.

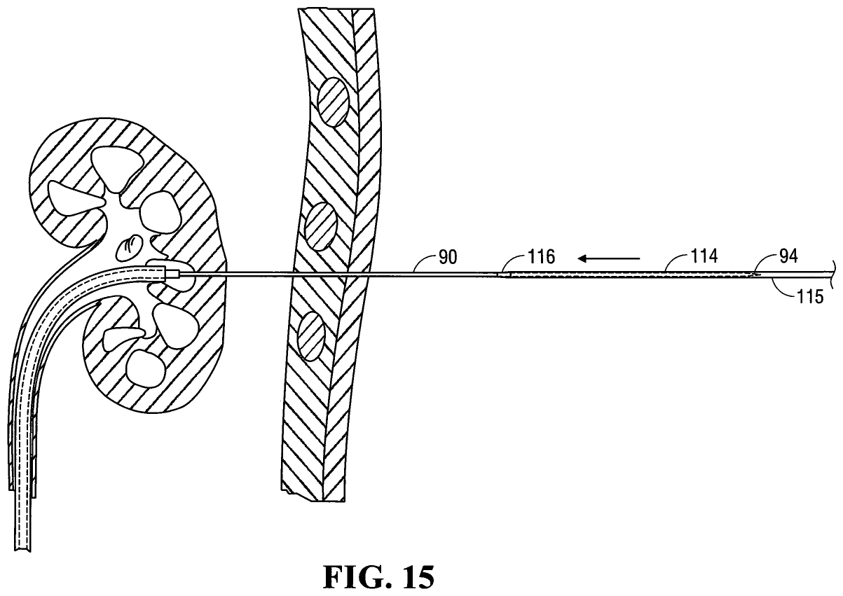

At this point, one of two methods/systems for insertion of the working wire can be utilized. One method is shown in FIGS. 9-14; an alternate method is shown in FIGS. 15-21. For convenience, the methods of FIG. 9-14 will be referred to herein as the “first” method and the method of FIG. 15-21 will be referred to as the “second” method.

The first method is a single catheter system and utilizes catheter 100 of FIG. 1F. As explained above, catheter 100 has a lumen 111 to receive the puncture wire 90. Catheter 110 (referred to herein as C1) as explained above has a distal tip 110 of a reduced diameter as it tapers inwardly in a distal direction to a diameter which preferably matches the outer diameter (OD) of the puncture wire. Stated another way, the smallest diameter of the lumen of the catheter 100 (which is at the distalmost tip) matches/equals (or alternatively is slightly larger than) the OD of the puncture wire 90; the diameter of the lumen proximal of lumen region 111a is greater than the OD of the puncture wire 90:
C1 lumen diameter at tip=OD puncture wire
C1 lumen diameter proximal of tip>OD puncture wire

In this way, the catheter 100 can be easily slid over the puncture wire and the tip of the catheter is not expanded during insertion of the puncture wire. The catheter tip has zero or close to zero clearance over the puncture wire. The narrower tip permits easy passage of the catheter tip through the flank fascia and renal capsule over the puncture wire, and the larger inner diameter (FIG. 1F) accommodates the larger diameter second wire.

The diameter of the lumen of catheter 100 closely matches (is substantially equal) the OD of the working (urology) wire 96, except at the tip:
C1 lumen diameter at tip<OD working wire
C1 lumen diameter proximal of tip=OD working wire

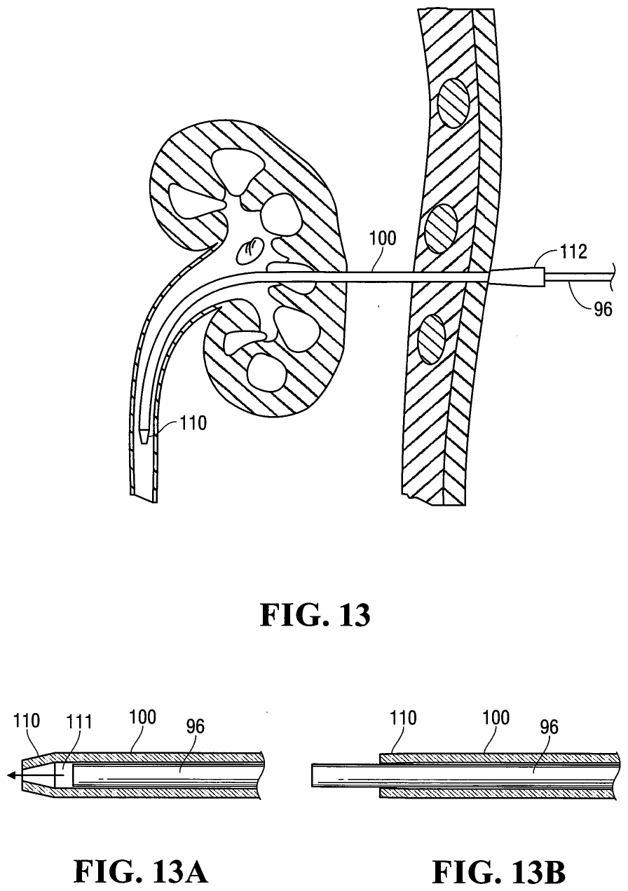

Therefore, when the urology wire 96 is inserted through the catheter 100, it will expand (dilate) the tip as described below in reference to FIGS. 13A and 13B.

The second method utilizes two catheters as shown in FIG. 1G. The first catheter 114 (referred to herein as CT1) has a lumen larger than the OD of the puncture wire and a lumen at the tip matching the OD of the puncture wire:
CT1 lumen diameter at tip=OD puncture wire
CT1 lumen diameter proximal of tip>OD puncture wire

In this manner, the puncture wire does not expand the distal tip of the first catheter 114.

However, the lumen of the first catheter 114 (CT1) is less than the OD of the working wire 96:
CT1 lumen diameter at tip<OD working wire
CT1 lumen diameter proximal of tip<OD working wire

In this manner, the first catheter 114 cannot receive (pass over) the working wire.

The second catheter 118 (referred to herein as CT2) of the second system has a lumen equal to the outer diameter of the working wire 96:
CT2 lumen diameter=OD working wire

In this manner, the second catheter 118 can receive the working wire 96.

Note CT2 can have in alternate embodiments a tapered tip less than the OD of the working wire which would be expanded by the working wire. In other embodiments, the lumen at the tapered tip and proximal of the tapered tip is greater than the OD of the working wire.

Turning to the first method of FIGS. 9-14, which utilizes the single lumen catheter, in the next step after the step in FIG. 6 where the puncture wire is advanced through the skin, the puncture wire 90 is extended further from the skin as shown in FIG. 9.

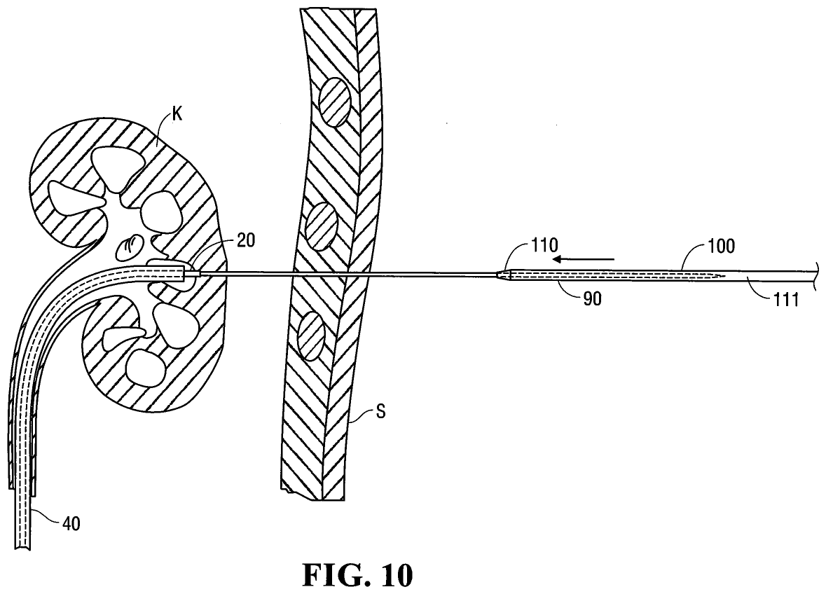

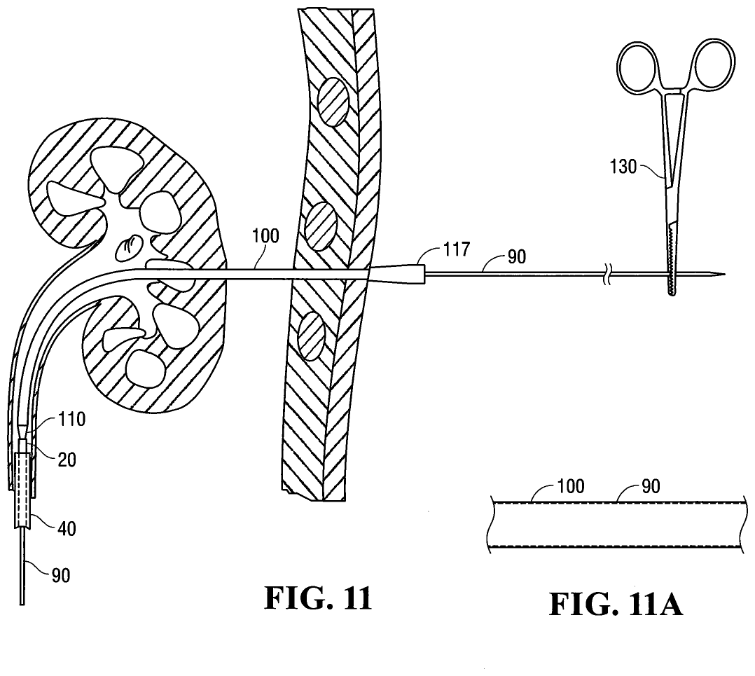

In the next step, illustrated in FIG. 10, the single lumen dilator/catheter 100 is inserted over the emergent section of the puncture wire 90 in a direction opposite to the direction of renal and flank puncture of the puncture wire 90. That is, the catheter 100 is inserted over the puncture wire tip and advanced through the skin in the antegrade direction of the arrow of FIG. 10. The tip of the catheter 100 as noted above is tapered to a diameter narrower than its lumenal diameter to have little or zero clearance over the puncture wire 90 (see FIG. 11A). The narrower tip permits easier passage of the catheter tip through the flank fascia and renal capsule over the puncture wire 90, and the larger inner diameter accommodates the larger diameter second wire inserted in subsequent steps. As shown in FIG. 11, the distal end of the puncture wire 90 exposed from the flank is held in position by a clamp 130 as the catheter 100 is advanced into the ureter.

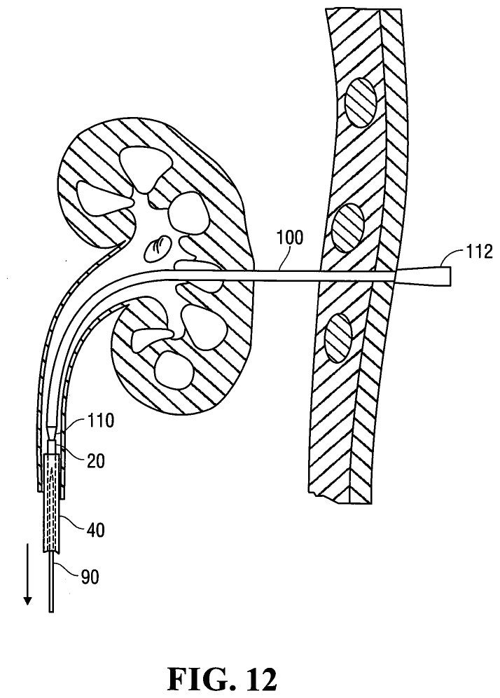

After the catheter 100 is advanced far enough over the puncture wire 90 into the patient to be positioned in the ureter as shown in FIG. 11, the puncture wire is removed (FIG. 12) in the opposite direction of its insertion. The uretero scope 40 and sheath 20 are also removed in the direction opposite the direction of insertion. Note the tip of the catheter 100 can abut the sheath and/or scope distal edge as it is advanced. The tip of the catheter may enter the open end of a ureteral access sheath which may be positioned in the ureter. Next, the larger diameter endourology working wire 96, is advanced through the open end of the catheter 100 exposed at the flank (FIG. 13), through the length of the catheter 100, and through the narrower tip, dilating the catheter tip 110 as the wire 96 is advanced through the tip 110 as shown in FIG. 13B. Note an acceptably low amount of force is required for advancing the second wire 96 to overcome the narrowed tip 110 of the catheter, thus allowing the larger wire to pass through this tip. Note the scope 40 and sheath 20 are removed prior to advancing the working wire 96 through catheter 100.

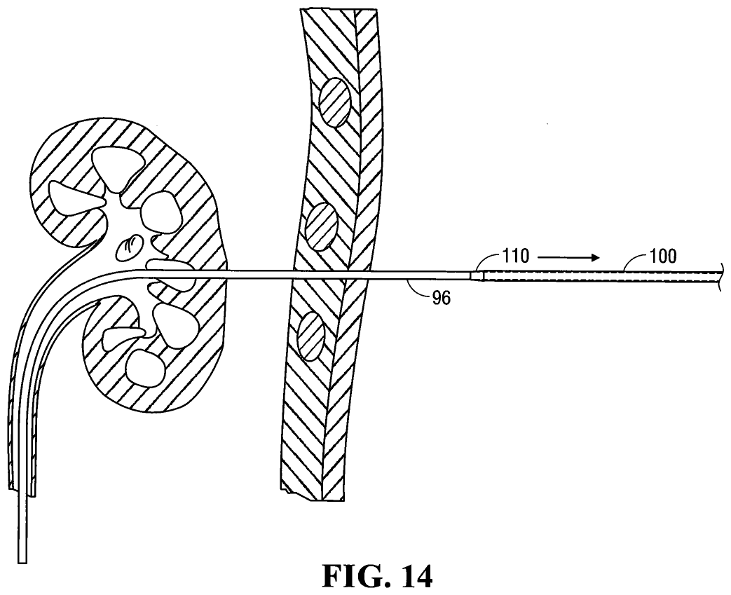

Next, the catheter 100 is removed in the direction opposite the direction of its insertion (in the retrograde direction of the arrow of FIG. 14), leaving the endourology wire 96 in the body extending into the ureter.

An alternate embodiment of the “wire exchange” system and method of the present invention is illustrated in FIGS. 15-21. In this embodiment, the system includes two catheters, preferably single lumen catheters. The method steps of FIGS. 3-6 are the same for this second method and differ from the first method starting at FIG. 15.

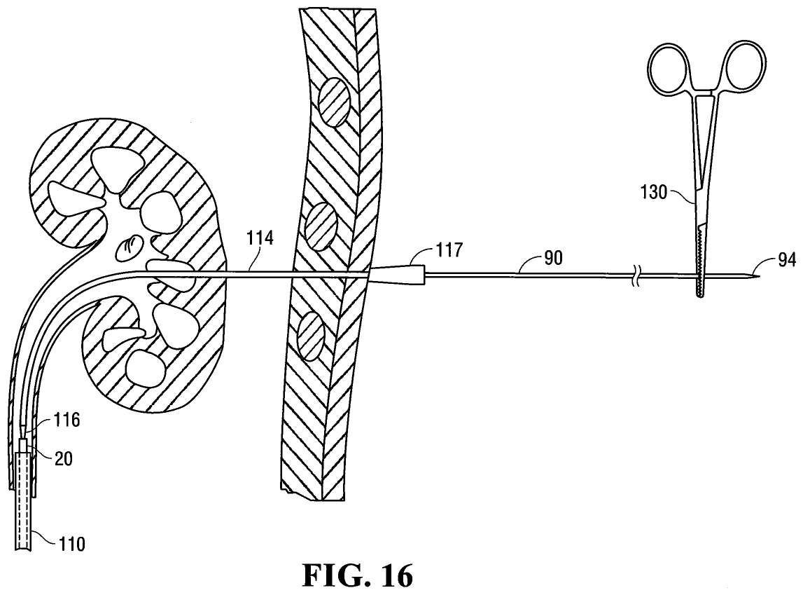

In FIG. 15, the first catheter 114 of FIG. 1G is advanced over the emergent section of the puncture wire 90 at the flank and advanced antegrade into the kidney and ureter for the purpose of dilating the tissues it passes through around the puncture wire (FIG. 16). Thus, its outer diameter is enough to sufficiently dilate the tissues around the puncture wire 90 to ease loading/passage of the second single lumen catheter over the puncture wire whose larger tip diameter is configured to accommodate the second larger diameter wire 96. The catheter tip can abut the tip of the scope and/or sheath during insertion.

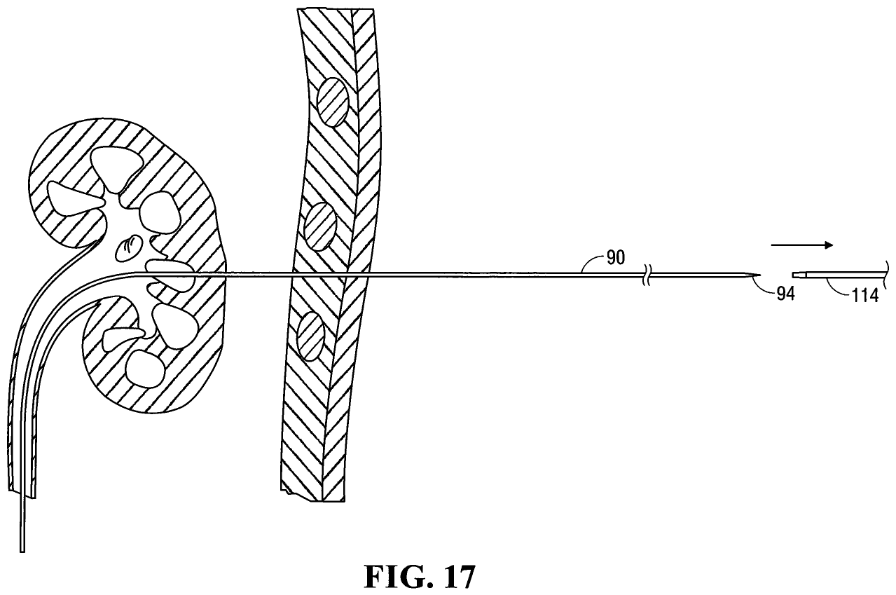

The first catheter 114 is then removed in a direction opposite its direction of insertion (FIG. 17), the second catheter 118 of FIG. 1G is loaded over the puncture wire 90. As noted above, the second catheter has an inner diameter along its length and tip large enough to accommodate the second, larger diameter nephrostomy working wire 96, typically 0.035″ or 0.038″, but at least 0.025″.

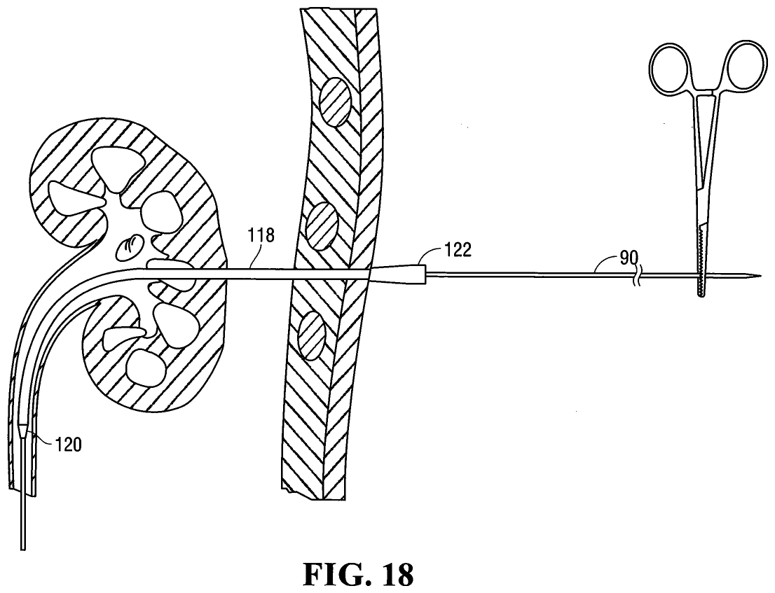

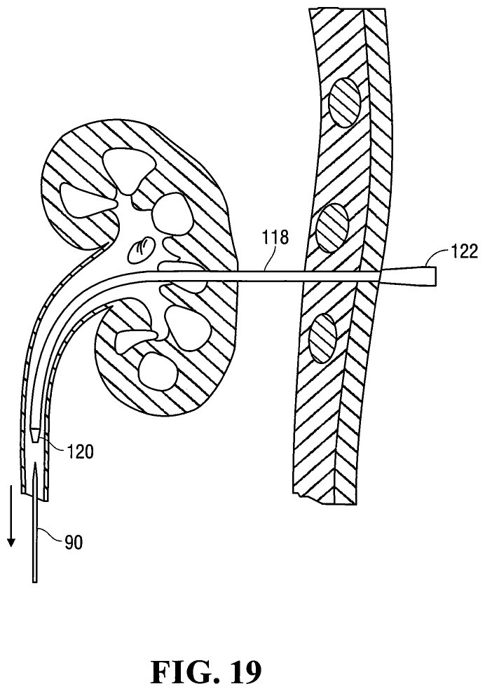

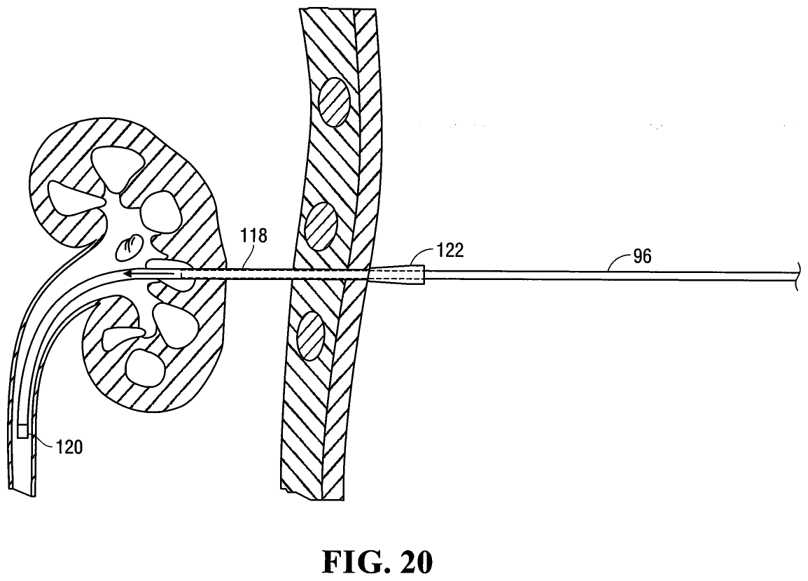

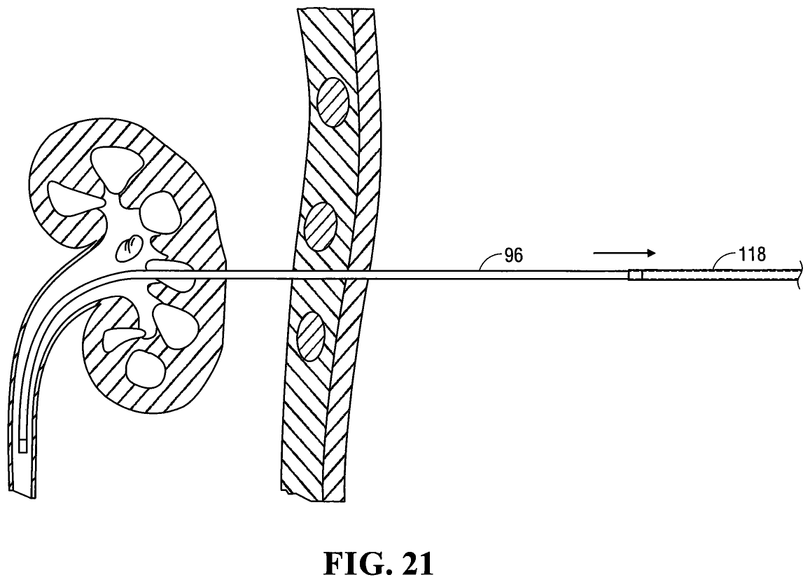

Once the second catheter 118 is advanced antegrade into the ureter (FIG. 18), the puncture wire 90 is removed in the opposite direction of its insertion (FIG. 19) and the second larger diameter working/endourology wire 96 is advanced through the open end of catheter 118 exposed at the flank, through the catheter (FIG. 20), and down the ureter. Note the scope 40 and sheath 20 are removed prior to advancing the working wire 96 through catheter 118. After insertion of wire 96, the second catheter 118 is removed in the direction opposite the direction of insertion as shown in FIG. 21, leaving the working wire 96 in position.

It is also contemplated that in an alternate embodiment, the puncture wire can be utilized without a protective sheath and inserted directly into the ureteroscope 40. The working channel 42 of the ureteroscope in this embodiment would thereby protect the puncture wire during insertion. This would reduce the number of components. Such sheathless puncture wire can be utilized with either method disclosed herein.

In these sheathless embodiments, the puncture wire can be locked to the operating (working) channel 42 of the ureteroscope 40 during insertion of the ureteroscope 40 into the calyx, and then the puncture wire released from locking engagement with the ureteroscope 40 to enable advancement distal of the end of the ureteroscope through the flank and skin. Such locking can be achieved with a vise lock or a locking mechanism similar to locking mechanism 60 described above, with the O-ring clamping on the puncture wire. Such embodiments enable a larger diameter puncture wire to be utilized, which could enable passage of a dilation balloon or other treatment devices directly over the puncture wire, thereby obviating the need for an exchange catheter and a second wire.

It is also contemplated that the characteristics of the puncture wire can be altered. For example, a coating can be applied to improve lubriciousness, and such coating can extend on a portion of or the length of the wire proximal of the tissue puncturing region. Coating with a low friction coefficient material could increase the wire caliber without significantly changing its handling properties. Preferably, the coating would not be applied to the distal 20-30 cm of the wire that is used to puncture the kidney, flank and skin.

The protective sheath for the puncture wire may be constructed to be thin walled to permit the entire puncture wire/protective sheath duo to maintain a small enough total diameter for passage through the working channel of ureteroscope. Use of materials such as PTFE (Teflon) or polyimide for sheath construction may have beneficial properties for this application.

The sheath may be constructed or post-processed to have enhanced visibility under ultrasound imaging. This may be achieved by any number of techniques, which may include but are not limited to placing a ceramic, graphite, Teflon, tungsten, Nitinol or platinum tip or outer coating on all or part of sheath or creating with or post-processing the sheath using laser or other abrasing or cutting technology to create small or microscopic grooves or indentations/dimples in the outer surface of the sheath to increase echogenicity.

It is also contemplated that all or part of the puncture wire and/or the exchange wire may be designed to have enhanced ultrasound visibility. This may allow for reduced radiation exposure during nephrostomy creation by allowing ultrasound guided confirmation of wire location during deployment. Options to achieve this include, but are not limited to the following: 1) Constructing the puncture wire and/or the exchange wire entirely of, or with a component of, a highly ultrasound-visible metal or other material. Examples include, but are not limited to, cobalt/chromium, graphite, Teflon, platinum or tungsten. These components may be mixed with stainless steel as an alloy or simply the distal tip of the wire can be made of these materials. 2) Coating the puncture wire and/or exchange wire with ceramic material, graphite, Teflon, tungsten, platinum, other metals or polymers, or material impregnated with microbubble technology such as glass microspheres, air microbubbles, or other adherent echogenic polymeric films. 3) The puncture wire and/or exchange wire may be constructed with or post-processed to create uneven surface(s) such as by brushing, lasering, creating indentations or cutting the outer surface of the wire. This would increase echogenicity of the wire.

Note the distal tip of the catheters can be radiopaque for localizing by fluoroscopy. To enhance imaging, additional regions of the catheter can be composed of radiopaque material, and even the entire length of the catheter.

The catheter may be constructed or post-processed to have enhanced visibility under ultrasound imaging. This may be achieved by any number of techniques, which may include but are not limited to placing a ceramic, graphite, Teflon, tungsten, Nitinol or platinum tip or outer coating on all or part of the catheter or creating with or post-processing the catheter using laser or other abrasing or cutting technology to create small or microscopic grooves or indentations/dimples in the outer surface of the catheter to increase echogenicity.

To create a nephrostogram to identify renal pelvis anatomy during balloon dilation of nephrostomy tract, the ureteral safety guidewire placed during the ureteroscopy portion of the procedure can be exchanged for a ureteric catheter through the urethra. Retrograde nephrostogram is performed through the ureteric catheter. A Foley catheter is placed in the bladder.

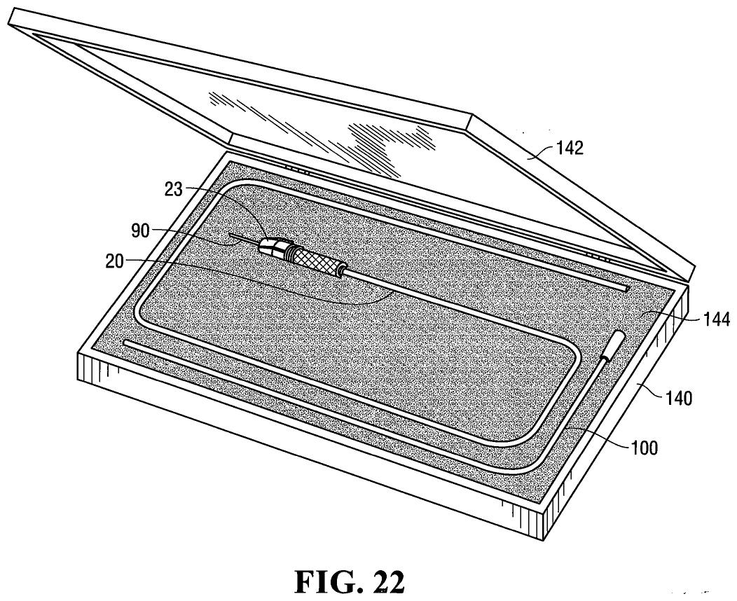

FIG. 22 illustrates one embodiment of a kit of the present invention. In this embodiment, kit 140 includes a cover 142 and packaging 144 with portions to receive the puncture wire 90/sheath 20 assembly as well as a space to receive the catheter 100. This kit can be utilized for the system/method utilizing a single catheter as in FIGS. 9-14. Also shown, the sheath/puncture wire length preferably is 2-3× the length of catheter 100. Note the kit could alternatively include any of the other puncture wires disclosed herein.

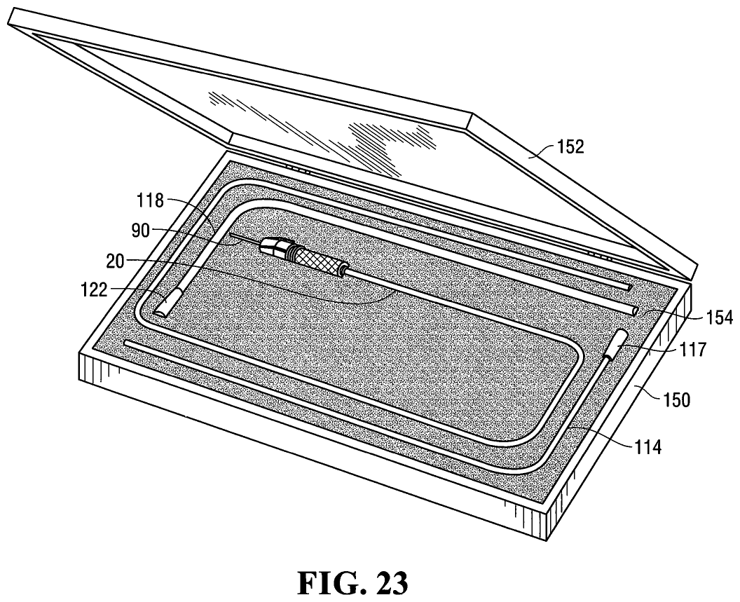

FIG. 23 illustrates an alternate embodiment of a kit of the present invention. In this embodiment, kit 150 includes a cover 152 and packaging 154 with portions to receive the puncture wire 90/sheath 20 assembly as well as a space to receive the catheter 114 and catheter 118. This kit can be utilized for the system/method utilizing two single lumen catheters as in FIG. 15-21. Note the kit could alternatively include any of the other puncture wires disclosed herein. As can be appreciated from the components in kit 150, the sheath/puncture wire 20 can have a length 2-3× the length of the catheters.

The sheath and catheters of kits 140 and 150 could also be packaged in a different configuration than the illustrated L-shape and in different locations than that shown.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

Although the apparatus and methods of the subject disclosure have been described with respect to preferred embodiments, those skilled in the art will readily appreciate that changes and modifications may be made thereto without departing from the spirit and scope of the present disclosure as defined by the appended claims.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the disclosure invention, terms such as “approximately,” “generally,” “substantially,” and the like should be understood to allow for variations in any numerical range or concept with which they are associated. For example, it is intended that the use of terms such as “approximately” and “generally” and “substantially” should be understood to encompass variations on the order of 25%, or to allow for manufacturing tolerances and/or deviations in design.

Although terms such as “first,” “second,” “third,” etc. may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A method for creating a tract in retrograde fashion for nephrostomy tube creation comprising the steps:
a) inserting a puncture wire and sheath in a first direction through a working channel in an ureteroscope, the puncture wire having a tissue penetrating tip shielded in a sheath;
b) advancing the puncture wire a first distance from the sheath and into a selected papilla and through a flank of a patient so the puncture wire has an emergent segment extending beyond the flank of the patient;
c) advancing a catheter over the emergent segment of the puncture wire outside the flank in a second direction opposite a direction of renal and flank puncture;
d) advancing the catheter into the flank in the second direction, the catheter having a lumen having a first section and a second section at a distal tip of the catheter, the first section having a first diameter and the second section having a second diameter smaller than the first diameter;
e) removing the puncture wire from the catheter; and
f) advancing a second wire in the second direction through the catheter into a ureter of the patient, the second wire having an outer diameter greater than an outer diameter of the puncture wire and greater than the second diameter of the second section of the lumen.

2. The method of claim 1, wherein the puncture wire is slidable within the sheath and releasingly lockingly engageable therein, and the puncture wire is released from the sheath prior to the step of advancing the puncture wire.

3. The method of claim 1, wherein the second catheter has a single lumen and the wire expands the distal tip of catheter when passed through the second section of the lumen.

4. The method of claim 1, wherein the puncture wire has a proximal segment with an outer diameter larger than a distal segment of the puncture wire to add stability to manual advancement of the catheter and prevent kinking of the wire during advancement of the catheter.

5. The method of claim 4, wherein the proximal segment is positioned outside an entry point to the ureteroscope when the puncture wire is inserted into the ureteroscope.

6. The method of claim 1, wherein the puncture wire has a distalmost puncture point, a first segment adjacent and proximal of the distalmost puncture point and a second segment proximal of the first segment, wherein the first segment has an outer diameter larger than an outer diameter of the puncture point and smaller than an outer diameter of the second segment to allow flexion of the first segment, and the second segment serves as a bridge over which the catheter is passed and limits kinking of the puncture wire during advancement of the catheter thereover.

7. The method of claim 1, wherein the puncture wire has a proximal segment with an outer diameter larger than a distal segment to prevent kinking during both advancement of the puncture wire through the ureteroscope and advancement of the catheter over the puncture wire in the second direction opposite the first direction of advancement through the ureteroscope.

8. The method of claim 1, wherein the distal tip of the first catheter is tapered to form second smaller diameter section.

9. The method of claim 8, wherein the step of advancing the second wire through the catheter expands the tapered distal tip of the catheter.

10. The method of claim 1, wherein the first section of the lumen is substantially equal to the outer diameter of the second wire.

11. A method for creating a tract in retrograde fashion for nephrostomy tube creation comprising the steps:
a) inserting a puncture wire having a tissue penetrating tip shielded in a sheath in a first direction through a working channel in an ureteroscope;
b) advancing the puncture wire from the sheath a first distance from the sheath and through a flank of a patient so the puncture wire has an emergent segment extending beyond the flank of the patient;
c) advancing a first catheter over the emergent segment of the puncture wire outside the flank in a second direction opposite a direction of renal and flank puncture;
d) advancing the first catheter into the flank to dilate tissue around the puncture wire, the first catheter having a distal tip having an inner diameter being a zero or close to zero fit over the puncture wire to ease passage into flank tissues;
e) removing the first catheter;
f) advancing a second catheter over the emergent section of the puncture wire outside the flank and advancing the second catheter into the flank and ureter in the second direction opposite the direction of renal and flank puncture;
g) removing the puncture wire leaving the second catheter in position in the ureter and outside the flank; and
h) advancing a second wire through the second catheter, the second wire having an outer diameter larger than an outer diameter of the puncture wire, wherein the second catheter has an inner diameter larger than the inner diameter of the first catheter.

12. The method of claim 11, wherein the second wire has an outer diameter greater than the inner diameter of the first catheter and the second catheter has an inner diameter equal to or larger than an outer diameter of the second wire.

13. The method of claim 11, wherein the first and second catheters each have a single lumen.

14. The method of claim 11, wherein the puncture wire is slidable within the sheath and releasingly lockingly engageable therein and the puncture wire is released from the sheath prior to the step (b).

15. The method of claim 11, wherein the first catheter has an inner tip diameter less than about 0.024 inches and the second catheter has an inner tip diameter larger than about 0.024 inches.

16. A system for creating a tract for nephrostomy tube creation comprising:
a) an exchange catheter having a tapered end forming a reduced diameter tip region, the tip region configured to be expanded by passage of a second wire having a larger outer diameter than the reduced diameter tip region;
b) a protective sheath; and
c) a puncture wire slidable within the sheath and releasably lockingly engageable with the sheath, the puncture wire insertable through a working channel of the ureteroscope, the puncture wire having a puncture tip and a thicker proximal segment at a point of advancement by a clinician above and outside an entry to the working channel of the ureteroscope, the proximal segment thicker than a distal segment adjacent the puncture tip, the thicker segment adding stability to advancement of the exchange catheter thereover and preventing kinking during advancement of the exchange catheter thereover.

17. The system of claim 16, further comprising the second wire, the second wire having an outer diameter larger than an outer diameter of the distal segment of the puncture wire and larger than an inner lumen of the reduced diameter tip region to expand the reduced diameter tip region when received therethrough.

18. The system of claim 16, wherein the exchange catheter has an inner tip diameter less than about 0.024 inches and the second catheter has an inner tip diameter larger than about 0.024 inches.

19. The system of claim 18, further comprising a second catheter, wherein the exchange catheter has an inner diameter less than outer diameter of the second wire and the second catheter has an inner diameter substantially equal to the outer diameter of the second wire.

20. The system of claim 16, wherein the system includes the second wire, wherein the reduced diameter tip region of the catheter is substantially equal to an outer diameter of the puncture wire and is less than an outer diameter of the second wire, and the exchange catheter has a lumen diameter proximal of the reduced diameter tip region which is greater than the outer diameter of the puncture wire and substantially equal to the outer diameter of the working wire.
